(19) United States
(12) Patent Application Publication
(10) Pub. No.: US 2018/0014719 A1
(43) Pub. Date: Jan. 18, 2018

(54) SCANNING ENDOSCOPE SYSTEM

(71) Applicant: OLYMPUS CORPORATION, Tokyo (JP)

(72) Inventors: Soichiro KOSHIKA, Tokyo (JP); Atsuyoshi SHIMAMOTO, Tokyo (JP); Masashi YAMADA, Tokyo (JP)

(73) Assignee: OLYMPUS CORPORATION, Tokyo (JP)

(21) Appl. No.: 15/698,906

(22) Filed: Sep. 8, 2017

Related U.S. Application Data
(63) Continuation of application No. PCT/JP2015/073153, filed on Aug. 18, 2015.

(30) Foreign Application Priority Data
Mar. 12, 2015 (JP) 2015-049801

(51) Int. Cl. A61B 1/00; A61B 1/07; G02B 23/26; G02B 23/24; G02B 6/036; G02B 26/10

(52) U.S. Cl. CPC A61B 1/00172; A61B 1/00167; G02B 23/26; G02B 23/2469; A61B 1/00006; A61B 1/07; G02B 26/103; G02B 6/036

(57) ABSTRACT

A scanning endoscope system includes an endoscope that includes an illumination fiber that is configured to guide illumination light for illuminating a subject and to emit the illumination light from an emitting end, and an actuator section that is configured to swing the emitting end of the illumination fiber according to a voltage or a current of an electrical signal that is applied to cause the illumination light to scan the subject, and a driver unit configured to apply, to the actuator section, the electrical signal that takes, as a drive frequency, a frequency at which an amount of change in amplitude at a time of swinging of the emitting end of the illumination fiber is at or below a predetermined value even when frequency characteristics of the amplitude are changed due to a change in a use condition of the endoscope.

### CROSS REFERENCE TO RELATED APPLICATION

This application is a continuation application of PCT/JP2015/073153 filed on Aug. 18, 2015 and claims benefit of Japanese Application No. 2015-049801 filed in Japan on Mar. 12, 2015, the entire contents of which are incorporated herein by this reference.

### BACKGROUND OF THE INVENTION

### 1. Field of the Invention

The present invention relates to a scanning endoscope system, and more particularly, to a scanning endoscope system which drives a fiber by an actuator, scans an object, and acquires an image.

### 2. Description of the Related Art

With regard to endoscopes in medical field, to reduce a burden on a subject, various techniques for reducing a diameter of an insertion section to be inserted into a body cavity of the subject are proposed. As an example of such techniques, a scanning endoscope system is known which causes light guided by an optical fiber to spirally scan an observation part, and which forms an image by receiving reflected light from the observation part.

According to such a scanning endoscope system, a fiber distal end is caused to draw a circle, by combining amplitude in each of an X direction and a Y direction with shifted phases. Therefore, the fiber distal end is desirably caused to vibrate in such a way as to draw a straight track in each of the X direction and the Y direction. Accordingly, a scanning endoscope system is proposed which uses, as a drive frequency allowing stable control of vibration amplitude of a fiber, a frequency which is a predetermined hertz away from a resonance frequency, instead of a frequency near the resonance frequency, based on an applied voltage to an actuator (for example, see Japanese Patent Application Laid-Open Publication No. 2014-198089).

When an environment surrounding the fiber is changed, frequency characteristics of the amplitude of the fiber shift to a lower frequency side or to a high frequency side. Particularly, a shift of the frequency characteristics is significant in a case where temperature around the fiber is changed. When the frequency characteristics are shifted, a change in the amplitude with respect to the frequency is great in a frequency domain around the resonance frequency, and the vibration amplitude of the fiber cannot be stably controlled. Accordingly, the fiber is desirably driven in a frequency band where a change in the amplitude is small even when the frequency characteristics are shifted due to a change in the environment, in a frequency domain away from the resonance frequency by a specific value. Also, the frequency characteristics of the amplitude are different for each scope, and thus, an optimal drive frequency domain is desirably set for each scope.

### SUMMARY OF THE INVENTION

A scanning endoscope system according to an aspect of the present invention includes a scanning section that includes a light guide section that is configured to guide illumination light for illuminating a subject and to emit the illumination light from an emitting end, and an actuator that is configured to swing the emitting end of the light guide section according to a voltage or a current of an electrical signal that is applied to cause the illumination light to scan the subject, and an application section configured to apply, to the actuator, the electrical signal that takes, as a drive frequency, a frequency at which an amount of change in amplitude at a time of swinging of the emitting end of the light guide section is at or below a predetermined value even when frequency characteristics of the amplitude are changed due to a change in a use condition of the scanning section.

### BRIEF DESCRIPTION OF THE DRAWINGS

FIG. 1 is a diagram showing an example of configuration of main parts of a scanning endoscope system according to an embodiment of the present invention;

FIG. 2 is a cross-sectional diagram for describing a configuration of an actuator section;

FIG. 3 is a diagram showing respective examples of signal waveforms of drive signals that are supplied to the actuator section;

FIG. 4 is a diagram showing an example of a spiral scan path extending from a center point A to an outermost point B;

FIG. 5 is a diagram showing an example of a spiral scan path extending from the outermost point B to the center point A;

FIG. 6 is a diagram showing a relationship between a drive frequency of the actuator section and amplitude of an emitting end portion of an illumination fiber;

FIG. 7 is a diagram describing a shift of frequency characteristics of the amplitude of the emitting end portion of the illumination fiber caused by a change in environment; and

FIG. 8 is a diagram showing another example of configuration of main parts of the scanning endoscope system according to the embodiment of the present invention.

### DETAILED DESCRIPTION OF THE PREFERRED EMBODIMENT(S)

Hereinafter, an embodiment will be described with reference to the drawings.

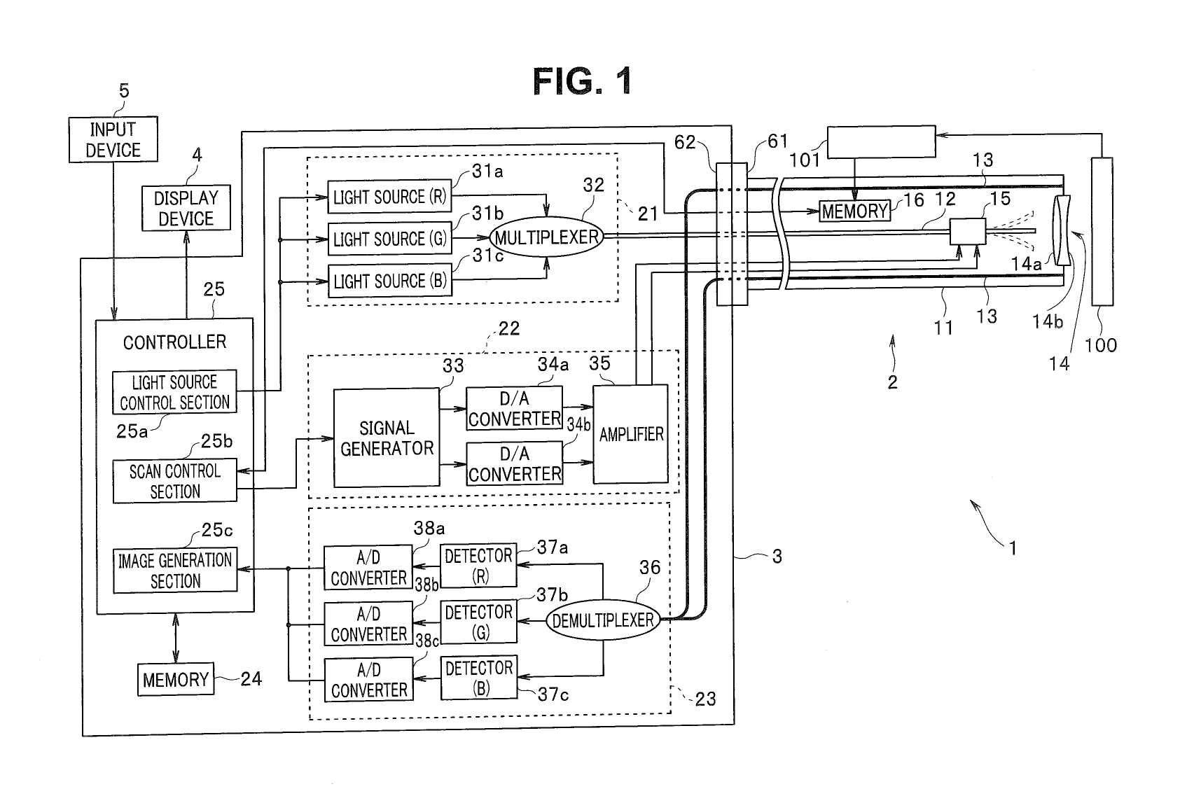

FIG. 1 is a diagram showing an example of configuration of main parts of a scanning endoscope system according to an embodiment of the present invention. As shown in FIG. 1, a scanning endoscope system 1 includes a scanning endoscope 2 which is inserted inside a body cavity of a subject, a main body device 3 to which the endoscope 2 can be connected, a display device 4 which is connected to the main body device 3, and an input device 5 allowing input of information and issuance of an instruction to the main body device 3, for example. The scanning endoscope system 1 also includes an amplitude detector 100, and a frequency characteristics calculation section 101.

The endoscope 2 as a scanning section includes an insertion section 11 formed to have an elongated shape insertable into a body cavity of a subject.

A connector section 61 for detachably connecting the endoscope 2 to a connector receiving section 62 of the main body device 3 is provided at a proximal end portion of the insertion section 11.

Although not shown, an electrical connector device which electrically connects the endoscope 2 and the main body device 3 is provided inside the connector section 61 and the connector receiving section 62. Also, although not shown, an optical connector device which optically connects the endoscope 2 and the main body device 3 is provided inside the connector section 61 and the connector receiving section 62.

Each of an illumination fiber 12 which is an optical fiber which guides illumination light supplied from a light source unit 21 of the main body device 3 to an illumination optical system 14, and a light receiving fiber 13 including at least one optical fiber which receives return light from an object and guides the light to a detection unit 23 of the main body device 3 is inserted through a part, of the inside of the insertion section 11, extending from the proximal end portion to a distal end portion.

An incident end portion, of the illumination fiber 12 as a light guide section, including a light incident surface is arranged in a multiplexer 32 which is provided inside the main body device 3. Also, an emitting end portion, of the illumination fiber 12, including a light emitting surface is arranged near a light incident surface of a lens 14a provided at the distal end portion of the insertion section 11.

An incident end portion, of the light receiving fiber 13, including a light incident surface is fixedly arranged around a light emitting surface of a lens 14b, at a distal end surface of the distal end portion of the insertion section 11. Also, an emitting end portion, of the light receiving fiber 13, including a light emitting surface is arranged at a demultiplexer 36 which is provided inside the main body device 3.

The illumination optical system 14 includes the lens 14a where illumination light from the light emitting surface of the illumination fiber 12 enters, and the lens 14b which emits the illumination light from the lens 14a to an object.

An actuator section 15 which is driven by a drive signal supplied from a driver unit 22 of the main body device 3 is provided at a mid-portion of the illumination fiber 12, on a distal end portion side of the insertion section 11.

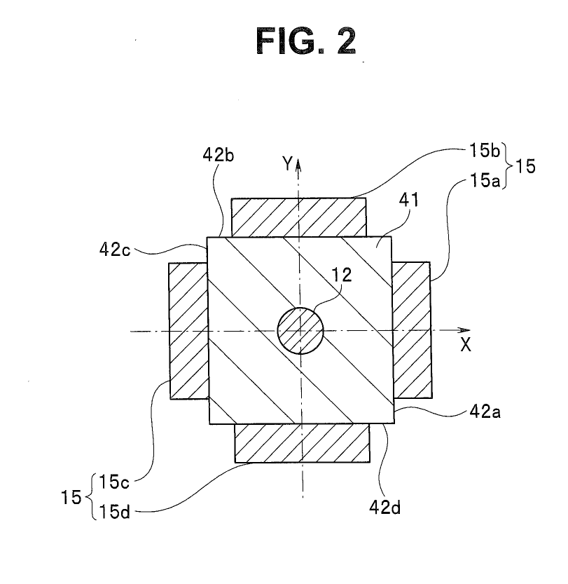

For example, the illumination fiber 12 and the actuator section 15 are arranged in a positional relationship as shown in FIG. 2 at a cross-section perpendicular to a longitudinal axis direction of the insertion section 11. FIG. 2 is a cross-sectional diagram for describing a configuration of the actuator section.

As shown in FIG. 2, a ferrule 41 as a joining member is arranged between the illumination fiber 12 and the actuator section 15. More specifically, the ferrule 41 is formed of zirconia (ceramics) or nickel, for example.

As shown in FIG. 2, the ferrule 41 is formed as a quadrangular prism, and includes side surfaces 42a and 42c that are perpendicular to an X-axis direction, which is a first axis direction orthogonal to the longitudinal axis direction of the insertion section 11, and side surfaces 42b and 42d that are perpendicular to a Y-axis direction, which is a second axis direction orthogonal to the longitudinal axis direction of the insertion section 11. Moreover, the illumination fiber 12 is fixedly arranged at the center of the ferrule 41. Note that the ferrule 41 may be formed to have a shape other than the quadrangular prism as long as the ferrule 41 has a columnar shape.

For example, as shown in FIG. 2, the actuator section 15 includes a piezoelectric element 15a that is arranged along the side surface 42a, a piezoelectric element 15b that is arranged along the side surface 42b, a piezoelectric element 15c that is arranged along the side surface 42c, and a piezoelectric element 15d that is arranged along the side surface 42d.

The piezoelectric element 15a-15d has a polarization direction which is individually set in advance, and is configured to expand or contract according to a drive voltage that is applied by a drive signal supplied from the main body device 3.

A non-volatile memory 16 for storing driving conditions of the actuator section 15 unique to each endoscope 2 is provided inside the insertion section 11. The driving conditions include setting conditions regarding a drive frequency of the actuator section 15 that is calculated, by a method described below, from the frequency characteristics of the amplitude of the illumination fiber 12. The driving conditions stored in the memory 16 are read by a controller 25 of the main body device 3 when the connector section 61 of the endoscope 2 and the connector receiving section 62 of the main body device 3 are connected and the power of the main body device 3 is turned on. Note that the setting conditions regarding the drive frequency of the actuator section 15 are stored in the memory 16 at an arbitrary timing before the timing of first use of the endoscope 2 by a user, such as at the time of manufacture of the endoscope 2.

The main body device 3 includes the light source unit 21, the driver unit 22, the detection unit 23, a memory 24, and the controller 25.

The light source unit 21 includes a light source 31a, a light source 31b, a light source 31c, and the multiplexer 32.

The light source 31a includes a laser light source, for example, and is configured to emit light in a red wavelength band (hereinafter referred to also as R light) to the multiplexer 32 when emitting light under control by the controller 25.

The light source 31b includes a laser light source, for example, and is configured to emit light in a green wavelength band (hereinafter referred to also as G light) to the multiplexer 32 when emitting light under control by the controller 25.

The light source 31c includes a laser light source, for example, and is configured to emit light in a blue wavelength band (hereinafter referred to also as B light) to the multiplexer 32 when emitting light under control by the controller 25.

The multiplexer 32 is configured to multiplex, and to supply to the light incident surface of the illumination fiber 12, the R light emitted by the light source 31a, the G light emitted by the light source 31b, and the B light emitted by the light source 31c.

The driver unit 22 as an application section is configured to generate a drive signal according to a drive voltage that is applied to the actuator section 15. Furthermore, the driver unit 22 includes a signal generator 33, D/A converters 34a and 34b, and an amplifier 35.

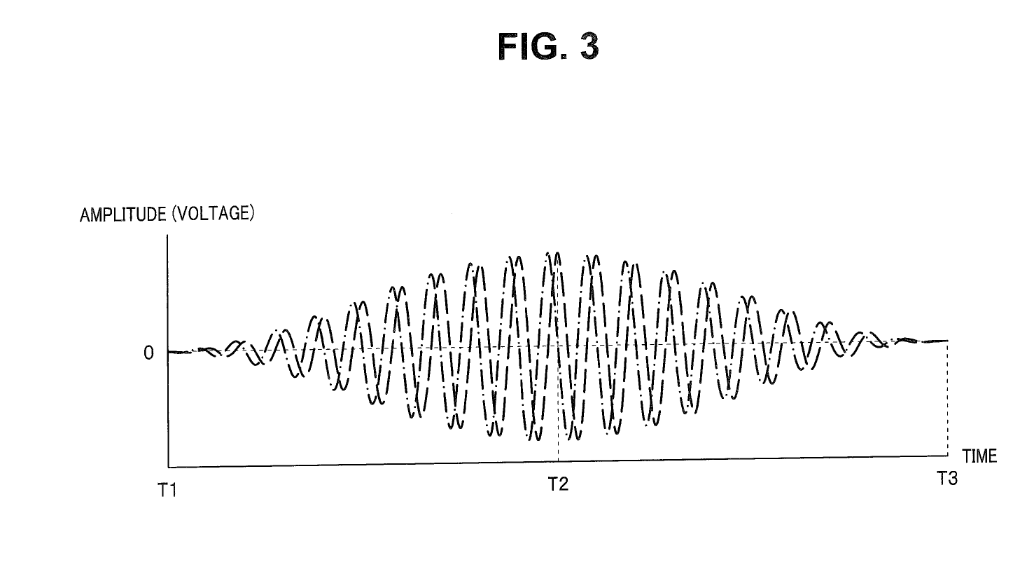

Under the control by the controller 25, the signal generator 33 generates, as a first drive signal for swinging the emitting end portion of the illumination fiber 12 in the X-axis direction, a voltage signal having a signal waveform that is obtained by applying predetermined modulation on a sine wave, as shown by a broken line in FIG. 3, and outputs the signal to the D/A converter 34a. Also, under the control by the controller 25, the signal generator 33 generates, as a second drive signal for swinging the emitting end portion of the illumination fiber 12 in the Y-axis direction, a voltage signal having a signal waveform having a phase that is shifted by 90 degrees from the first drive signal, as shown by a dashed-dotted line in FIG. 3, and outputs the signal to the D/A converter 34b. FIG. 3 is a diagram showing respective examples of the signal waveforms of the drive signals that are supplied to the actuator section.

The D/A converter 34a is configured to convert the digital first drive signal outputted from the signal generator 33 into an analog first drive signal, and to output the signal to the amplifier 35.

The D/A converter 34b is configured to convert the digital second drive signal outputted from the signal generator 33 into an analog second drive signal, and to output the signal to the amplifier 35.

The amplifier 35 is configured to amplify the first and the second drive signals outputted from the D/A converters 34a and 34b, and to output the signals to the actuator section 15.

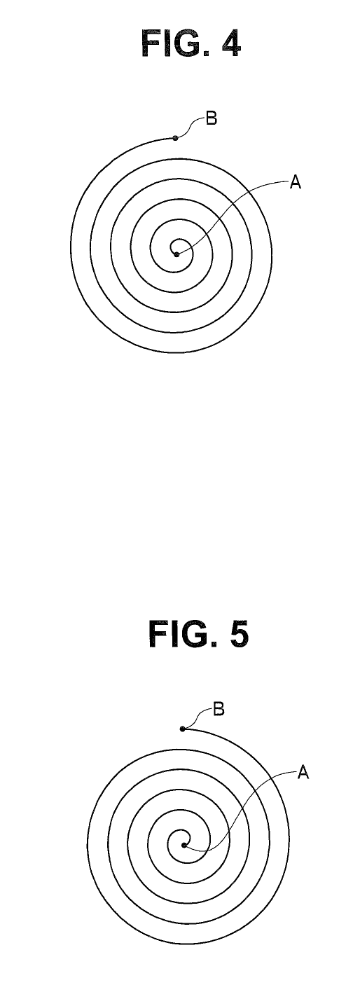

For example, the emitting end portion of the illumination fiber 12 is swung in a spiral manner by application of the drive voltage according to the first drive signal having a signal waveform as shown by the broken line in FIG. 3 to the piezoelectric elements 15a and 15c of the actuator section 15 and by application of the drive voltage according to the second drive signal having a signal waveform as shown by the dashed-dotted line in FIG. 3 to the piezoelectric elements 15b and 15d of the actuator section 15, and a surface of an object is scanned, due to such swinging, along a spiral scan path as shown in FIGS. 4 and 5. FIG. 4 is a diagram showing an example of a spiral scan path extending from a center point A to an outermost point B. FIG. 5 is a diagram showing an example of a spiral scan path extending from the outermost point B to the center point A.

More specifically, first, at a time T1, illumination light is radiated on a position, on a surface of an object, corresponding to the center point A of radiation position of illumination light. Then, as the amplitude (voltage) of the first and the second drive signals is increased from the time T1 to a time T2, the radiation position of the illumination light on the surface of the object is displaced from the center point A toward the outside to draw a first spiral scan path, and when the time T2 is reached, the illumination light is radiated on the outermost point B of the radiation position of the illumination light on the surface of the object. Then, as the amplitude (voltage) of the first and the second drive signals is reduced from the time T2 to a time T3, the radiation position of the illumination light on the surface of the object is displaced from the outermost point B toward the inside to draw a second spiral scan path, and when the time T3 is reached, the illumination light is radiated on the center point A on the surface of the object.

That is, the actuator section 15 is configured to be able to displace the radiation position of the illumination light emitted to an object through the emitting end portion of the illumination fiber 12 along the spiral scan path shown in FIGS. 4 and 5 by swinging the emitting end portion based on the first and the second drive signals supplied from the driver unit 22. Also, the amplitude of the first and the second drive signals supplied from the driver unit 22 to the actuator section 15 is maximized at the time T2 or around the time T2. Furthermore, in the example of the spiral scan path in FIGS. 4 and 5, the scan range of the endoscope 2 is shown as a region which includes the outermost point B of the spiral scan path and which is on the inside of the outermost circumference path, and is changed according to the size of the maximum amplitude of the drive signals supplied to the actuator section 15.

The detection unit 23 includes the demultiplexer 36, detectors 37a, 37b and 37c, and A/D converters 38a, 38b and 38c.

The demultiplexer 36 includes a dichroic mirror or the like, and is configured to separate return light emitted from the light emitting surface of the light receiving fiber 13 into light of each of color components R (red), G (green) and B (blue), and to emit the light to the detectors 37a, 37b and 37c.

The detector 37a includes an avalanche photodiode, for example, and is configured to detect intensity of R light outputted from the demultiplexer 36, to generate an analog R signal according to the detected intensity of the R light, and to output the signal to the A/D converter 38a.

The detector 37b includes an avalanche photodiode, for example, and is configured to detect intensity of G light outputted from the demultiplexer 36, to generate an analog G signal according to the detected intensity of the G light, and to output the signal to the A/D converter 38b.

The detector 37c includes an avalanche photodiode, for example, and is configured to detect intensity of B light outputted from the demultiplexer 36, to generate an analog B signal according to the detected intensity of the B light, and to output the signal to the A/D converter 38c.

The A/D converter 38a is configured to convert the analog R signal outputted from the detector 37a into a digital R signal, and to output the signal to the controller 25.

The A/D converter 38b is configured to convert the analog G signal outputted from the detector 37b into a digital G signal, and to output the signal to the controller 25.

The A/D converter 38c is configured to convert the analog B signal outputted from the detector 37c into a digital B signal, and to output the signal to the controller 25.

The memory 24 stores, as control information which is used at the time of control of the main body device 3, information including various parameters for causing the light sources 31a-31c to emit light and parameters such as amplitude or a phase difference for identifying the signal waveforms in FIG. 3, for example.

The controller 25 is configured by an integrated circuit such as an FPGA (field programmable gate array). Also, the controller 25 is configured to be able to detect whether the insertion section 11 is electrically connected to the main body device 3, by detecting a connection state of the connector section 61 at the connector receiving section 62 through a signal line or the like, not shown. Moreover, the controller 25 includes a light source control section 25a, a scan control section 25b, and an image generation section 25c.

For example, the light source control section 25a is configured to control the light source unit 21 such that the light sources 31a-31c simultaneously emit light, based on the control information read from the memory 24.

For example, the scan control section 25b as a setting section is configured to read drive frequency conditions of the actuator section 15 stored in the memory 16 as described above, when the connector section 61 of the endoscope 2 and the connector receiving section 62 of the main body device 3 are connected and the power of the main body device 3 is turned on, for example. The driver unit 22 is controlled such that a drive signal having a signal waveform as shown in FIG. 3 is generated, for example, based on driving conditions unique to the endoscope 2, including the drive frequency conditions read from the memory 16, and the control information read from the memory 24.

For example, the image generation section 25c is configured to generate an observation image for one frame by detecting a closest scan path based on the signal waveforms of drive signals generated under the control by the scan control section 25b, identifying a pixel position, in a raster scan format, corresponding to the radiation position of illumination light on the detected scan path, and mapping brightness values indicated by the digital signals outputted from the detection unit 23 to the identified pixel position, and to sequentially output generated observation images for respective frames to the display device 4. Also, the image generation section 25c is configured to be able to perform a process of displaying, as an image, a predetermined text or the like on the display device 4.

The display device 4 includes a monitor or the like, and is configured to be able to display an observation image that is outputted from the main body device 3.

The input device 5 includes a keyboard or a touch panel, for example. Note that the input device 5 may be configured as a device separate from the main body device 3, or may be configured as an interface that is integrated with the main body device 3.

The amplitude detector 100 is configured to detect a swing width (amplitude) of the emitting end portion of the illumination fiber 12 when the actuator section 15 is driven and the illumination fiber 12 is caused to swing. As the amplitude detector 100, a general amplitude detection sensor, such as a position sensitive detector (PSD), may be used. The amplitude of the emitting end portion of the illumination fiber 12 detected by the amplitude detector 100 is outputted to the frequency characteristics calculation section 101.

The frequency characteristics calculation section 101 calculates the drive frequency domain of the actuator section 15 where amplitude which is stable regardless of a change in the ambient environment of the endoscope 2 can be obtained, based on a relationship between the amplitude of the emitting end portion of the illumination fiber 12 inputted from the amplitude detector 100 and the drive frequency of the actuator section 15. In the following, a calculation method of the drive frequency domain will be described.

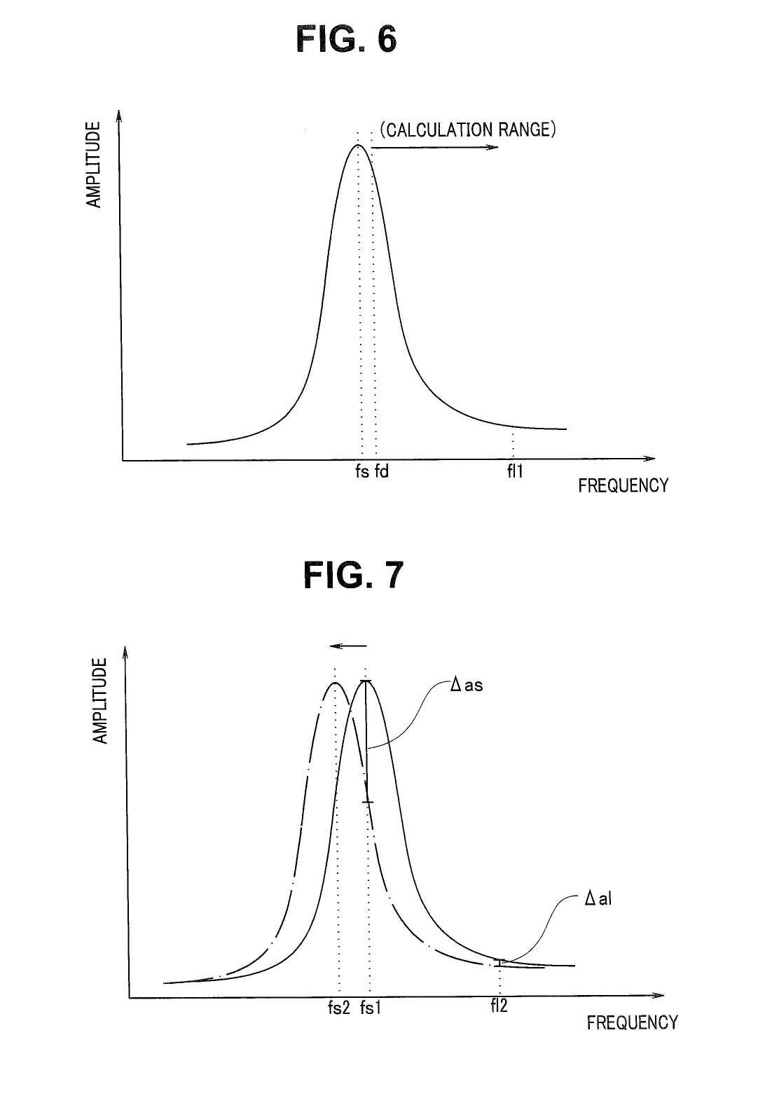

First, a method of calculating the drive frequency domain by using an inclination of the frequency characteristics of the amplitude of the emitting end portion of the illumination fiber 12 will be described with reference to FIG. 6. FIG. 6 is a diagram showing a relationship between the drive frequency of the actuator section and the amplitude of the emitting end portion of the illumination fiber. As shown in FIG. 6, the amplitude of the emitting end portion of the illumination fiber 12 takes a maximum value when the drive frequency of the actuator section 15 is at a resonance frequency fs. The amplitude of the emitting end portion is drastically reduced when the drive frequency is separated away from the resonance frequency fs, and the amplitude takes an approximately constant value in a frequency domain where the drive frequency is separated from the resonance frequency fs by a predetermined value or more.

In the frequency domain where the amplitude takes an approximately constant value, even if the frequency characteristics are shifted due to a change in the environment, such as temperature or humidity, of the illumination fiber 12, the amplitude is only slightly changed before and after the shift. Accordingly, an upper limit value (a first threshold) of the inclination of the frequency characteristics is set in advance based on, for example, an allowable amount of change in the amplitude before and after a shift, and a frequency fl1 at which the inclination becomes equal to the first threshold, according to the frequency characteristics of the amplitude of the emitting end portion of the illumination fiber 12 inputted from the frequency characteristics calculation section 101, is determined. Then, in the case of driving the actuator section 15 at a high frequency, a frequency domain taking the frequency fl1 as a lower limit is set as the drive frequency domain. Note that an upper limit value of the inclination of the frequency characteristics is desirably substantially zero.

Also at a frequency near the resonance frequency fs, the amplitude of the emitting end portion of the illumination fiber 12 takes an approximately constant value, and thus, the inclination is substantially zero. Accordingly, frequency characteristics of a domain within a predetermined value (for example, about 20 Hz) from the resonance frequency fs is not used for calculation of the frequency fl, and the frequency fl1 is calculated by using the frequency characteristics of a frequency which is separated from the resonance frequency fs by a predetermined value or more. For example, as shown in FIG. 6, in a case where the actuator section 15 is to be driven at a high frequency, the frequency fl1 is calculated by using the frequency characteristics of a range at and above a frequency fd which is on a higher frequency side than the resonance frequency fs by a predetermined value (for example, about 20 Hz).

Furthermore, when a slight external vibration is transmitted to the illumination fiber 12 during measurement of the frequency characteristics, a noise may become contained in the waveform. When a noise is contained, the amplitude of a frequency where the noise occurred is increased compared to a normal case, and a sharp peak appears at the frequency. If the inclination of frequency characteristics with a noise is calculated, the frequency at the peak portion of the noise also becomes substantially zero, and a correct value may not be obtained as the frequency fl1.

Accordingly, in the case of determining the frequency fl1 at which the inclination of the frequency characteristics is equal to the first threshold, the continuity of the inclination of the frequency characteristics is desirably taken into consideration. That is, in a case where the inclination of the frequency characteristics in a specific frequency range is continuously at or below the first threshold, a frequency closest to the resonance frequency, among frequencies where the inclination of the frequency characteristics is at or below the first threshold, is calculated as the frequency fl1.

Note that, in the case of driving the actuator section 15 at a low frequency, a frequency fl1′, on a lower frequency side of the resonance frequency fs, at which the inclination is equal to the first threshold is calculated, and a frequency domain taking the frequency fl1′ as the upper limit is set as the drive frequency domain.

Next, a method of calculating the drive frequency domain by using an amount of shift of the amplitude of the emitting end portion of the illumination fiber 12 will be described with reference to FIG. 7. As changes in the environmental which cause the frequency characteristics of the amplitude to be shifted, a change in temperature and a change in the humidity may be cited, for example. In the present case, a method of calculating the drive frequency domain will be described while citing, as an example, a shift of the frequency characteristics occurring when a change in the environment is a change in temperature.

FIG. 7 is a diagram describing a shift of the frequency characteristics of the amplitude of the emitting end portion of the illumination fiber caused by a change in environment. In FIG. 7, the frequency characteristics of the amplitude of the emitting end portion of the illumination fiber at a normal temperature are shown by a solid line. Also, the frequency characteristics of the amplitude of the emitting end portion of the same illumination fiber which is exposed to a high temperature environment are shown by a dashed-dotted line. Note that an approximate normal room temperature (for example, about 25 degrees Celsius) is taken as the normal temperature, which is a temperature of an approximate temperature inside the body of a subject (for example, about 37 degrees Celsius).

The frequency characteristics of the amplitude of the emitting end portion of the illumination fiber 12 tend to shift to the low-frequency side when the ambient environment changes from a normal temperature to a high temperature. For example, as shown in FIG. 7, when the ambient environment reaches a high temperature, frequency characteristics at a resonance frequency fs1 at a normal temperature tend to shift to the low-frequency side, and the resonance frequency shifts to a frequency fs2 of a shorter wavelength than the frequency fs1. That is, the amplitude at the same frequency is changed before and after the change in the environment.

An amount of change Δa in the amplitude caused by a change in the environment is smaller in a frequency domain which is away from the resonance frequency fs than in a frequency domain near the resonance frequency fs. For example, as shown in FIG. 7, an amount of change Δas in the amplitude at the resonance frequency fs1 at a normal temperature is great, being about 30% of the amplitude at the normal temperature. On the other hand, an amount of change Δa1 in the amplitude at a frequency fl in a frequency domain which is away from the resonance frequency fs1 is within a small value of about several percent of the amplitude at the normal temperature.

When the amplitude of the emitting end portion of the illumination fiber 12 is changed, the scan range of illumination light is changed, and thus, an angle of view of an image obtained from the light receiving fiber 13 is also changed. Generally, a target value is set for the angle of view. Accordingly, an upper limit (a second threshold) of an allowable proportion of the amount of change in the amplitude is set in advance based on the target value, and a frequency fl2 at which the proportion of the amount of change Δa1 in the amplitude before and after a change in the environment is equal to the second threshold, according to the frequency characteristics of the amplitude of the emitting end portion of the illumination fiber 12 inputted from the frequency characteristics calculation section 101, is determined. Then, in the case of driving the actuator section 15 at a high frequency, a frequency domain taking the frequency fl2 as the lower limit is set as the drive frequency domain.

For example, in a case where a change in the amplitude of up to 5% is allowed to achieve the target value of the angle of view, a frequency fl2 at which the proportion of the amount of change Δa1 in the amplitude before and after a change in the environment is 5% with respect to the amplitude at the normal temperature is calculated. Then, a frequency domain taking the frequency fl2 as the lower limit is set as the drive frequency domain. Note that, in the case of driving the actuator section 15 at a low frequency, a frequency fl2′ at which the proportion of the amount of change Δa in the amplitude before and after a change in the environment is 5% with respect to the amplitude at the normal temperature is calculated on the lower frequency side of the resonance frequency fs1, and a frequency domain taking the frequency fl2′ as the upper limit is set as the drive frequency domain.

Note that the frequency characteristics calculation section 101 may be a general-purpose computer, such as a personal computer.

Next, an operation, of the scanning endoscope system 1 having the configuration as described above, for a case of calculating a drive frequency domain by using the inclination of the frequency characteristics of the amplitude of the emitting end portion of the illumination fiber 12, and recording the drive frequency domain in the memory 16 will be described.

For example, at the time of manufacture of the endoscope 2, a factory worker connects each part of the optical scanning observation system 1 and switches on the power in a state where the endoscope 2 is placed in an environment where temperature of the actuator section 15 is at a predetermined temperature TEM.

Note that the predetermined temperature TEM is a temperature in the range of normal temperature, such as 25 degrees Celsius.

Then, the factory worker instructs the controller 25 to start scanning by the endoscope 2, by operating a scan start switch (not shown) of the input device 5, for example.

When the scan start switch of the input device 5 is operated, the scan control section 25b controls the driver unit 22 such that a drive signal having a predetermined drive voltage and a predetermined drive frequency is generated, based on control information read from the memory 24. Note that the predetermined drive voltage is a drive voltage according to which the angle of view is within an allowable range and the amplitude of the emitting end portion of the illumination fiber 12 is within a range allowing detection by the amplitude detector 100 even when the actuator section 15 is driven at the resonance frequency fs. Also, the predetermined drive frequency is a drive frequency according to which the frequency is continuously changed in a range from a frequency which is lower than the resonance frequency fs by a predetermined value to a frequency which is higher than the resonance frequency fs by a predetermined value. For example, in a case where the resonance frequency is 9000 Hz, control for generating a drive signal according to which the drive frequency of the actuator section 15 changes in the range of 8500 Hz to 9500 Hz is inputted to the driver unit 22.

The amplitude detector 100 detects the swing width (amplitude) of the emitting end portion of the illumination fiber 12 in the X-axis direction and the Y-axis direction, and outputs the detected amplitude to the frequency characteristics calculation section 101.

The frequency characteristics calculation section 101 calculates the frequency characteristics of the amplitude by using the amplitude of the emitting end portion of the illumination fiber 12 inputted from the amplitude detector 100 and the drive frequency of the actuator section 15. A frequency at which the inclination of the calculated frequency characteristics is at the first threshold that is set in advance is determined. In the case where the determined frequency is higher than the resonance frequency fs, the frequency is stored in the memory 16 as the lower limit value of the drive frequency of the actuator section 15 at the time of high-frequency driving. In the case where the determined frequency is lower than the resonance frequency fs, the frequency is stored in the memory 16 as the upper limit value of the drive frequency of the actuator section 15 at the time of low-frequency driving. Then, the calculated drive frequency domain is stored in the memory 16, and then, a notice to the effect that calculation and recording of the drive frequency domain are complete is outputted to the scan control section 25b.

The scan control section 25b controls the image generation section 25c such that a text or the like is displayed by the display device 4 so as to notify the factory worker of the notice, outputted from the frequency characteristics calculation section 101, to the effect that calculation and recording of the drive frequency domain are complete. Calculation and recording, in the memory 16, of the drive frequency domain of the actuator section 15 using the inclination of the frequency characteristics of the amplitude of the emitting end portion of the illumination fiber 12 at the predetermined temperature TEM are completed by a series of operations described above.

Next, an operation, of the scanning endoscope system 1 having the configuration as described above, for a case of calculating a drive frequency domain by using an amount of shift of the amplitude of the emitting end portion of the illumination fiber 12, and recording the drive frequency domain in the memory 16 will be described.

For example, at the time of manufacture of the endoscope 2, a factory worker connects each part of the optical scanning observation system 1 and switches on the power in a state where the endoscope 2 is placed in an environment where the temperature of the actuator section 15 is at a predetermined temperature TEM. Note that the predetermined temperature TEM is a temperature in the range of normal temperature, such as 25 degrees Celsius.

Then, the factory worker instructs the controller 25 to start scanning by the endoscope 2, by operating a scan start switch (not shown) of the input device 5, for example.

When the scan start switch of the input device 5 is operated, the scan control section 25b controls the driver unit 22 such that a drive signal having a predetermined drive voltage and a predetermined drive frequency is generated, based on control information read from the memory 24. Note that the predetermined drive voltage is a drive voltage according to which the angle of view is within an allowable range and the amplitude of the emitting end portion of the illumination fiber 12 is within a range allowing detection by the amplitude detector 100 even when the actuator section 15 is driven at the resonance frequency fs. Also, the predetermined drive frequency is a drive frequency according to which the frequency is continuously changed in a range from a frequency which is lower than the resonance frequency fs by a predetermined value to a frequency which is higher than the resonance frequency fs by a predetermined value. For example, in a case where the resonance frequency is 9000 Hz, control for generating a drive signal according to which the drive frequency of the actuator section 15 changes in the range of 8500 Hz to 9500 Hz is inputted to the driver unit 22.

The amplitude detector 100 detects the swing width (amplitude) of the emitting end portion of the illumination fiber 12 in the X-axis direction and the Y-axis direction, and outputs the detected amplitude to the frequency characteristics calculation section 101. The frequency characteristics calculation section 101 calculates the frequency characteristics of the amplitude at the temperature TEM by using the amplitude of the emitting end portion of the illumination fiber 12 inputted from the amplitude detector 100 and the drive frequency of the actuator section 15.

Next, the factory worker places the endoscope 2 in an environment where the temperature of the actuator section 15 is at a predetermined temperature TEB. Note that the predetermined temperature TEB is a temperature in the range of high temperature, such as 37 degrees Celsius.

The amplitude detector 100 subsequently detects the swing width (amplitude) of the emitting end portion of the illumination fiber 12 in the X-axis direction and the Y-axis direction, and outputs the detected amplitude to the frequency characteristics calculation section 101. The frequency characteristics calculation section 101 calculates the frequency characteristics of the amplitude at the temperature TEB by using the amplitude of the emitting end portion of the illumination fiber 12 inputted from the amplitude detector 100 and the drive frequency of the actuator section 15.

The frequency characteristics calculation section 101 determines a frequency at which the proportion of the amount of change Δa in the amplitude becomes equal to the second threshold, by using the frequency characteristics of the amplitude at the temperature TEM and the frequency characteristics of the amplitude at the temperature TEB. In the case where the determined frequency is higher than the resonance frequency fs, the frequency is stored in the memory 16 as the lower limit value of the drive frequency of the actuator section 15 at the time of high-frequency driving. In the case where the determined frequency is lower than the resonance frequency fs, the frequency is stored in the memory 16 as the upper limit value of the drive frequency of the actuator section 15 at the time of low-frequency driving. Then, the calculated drive frequency domain is stored in the memory 16, and then, a notice to the effect that calculation and recording of the drive frequency domain are complete is outputted to the scan control section 25b.

The scan control section 25b controls the image generation section 25c such that a text or the like is displayed by the display device 4 so as to notify the factory worker of the notice, outputted from the frequency characteristics calculation section 101, to the effect that calculation and recording of the drive frequency domain are complete. Calculation and recording, in the memory 16, of the drive frequency domain of the actuator section 15 using the inclination of the frequency characteristics of the amplitude of the emitting end portion of the illumination fiber 12 at the predetermined temperature TEM are completed by a series of operations described above.

As described above, according to the present example, frequency characteristics of the amplitude of the emitting end portion of the illumination fiber 12 are acquired before use of the endoscope 2, for example, and a frequency domain where the inclination is at or below the first threshold, or a frequency domain where the proportion of the amount of change in the amplitude when the frequency characteristics are shifted is at or below the second threshold is recorded in the memory 16 as the drive frequency domain of the actuator section 15. When actually using the endoscope 2, the actuator section 15 is driven at a frequency in the drive frequency domain recorded in the memory 16 so as to enable stable control of the amplitude of the emitting end portion of the illumination fiber 12 even when the use environment of the endoscope 2 is changed.

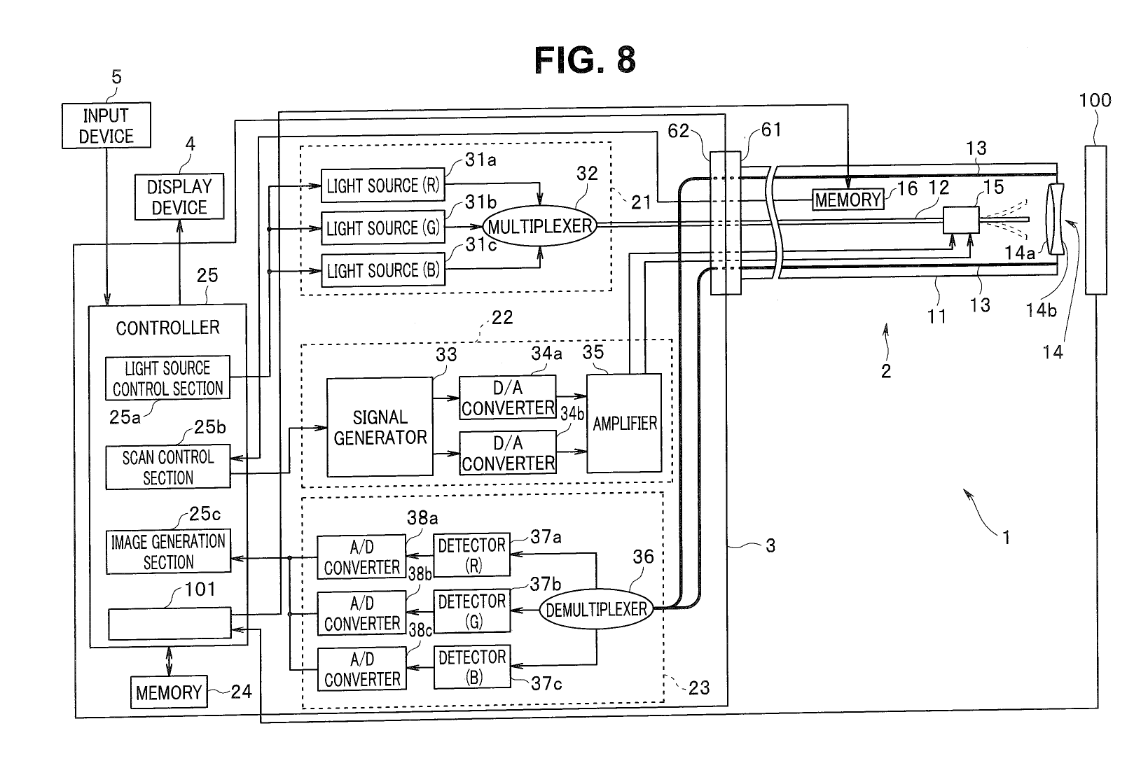

Note that FIG. 8 is a diagram showing another example of configuration of main parts of the scanning endoscope system according to the embodiment of the present invention. In the embodiment described above, the frequency characteristics calculation section 101 is arranged separately from the endoscope 2 and the main body device 3, but the frequency characteristics calculation section 101 may be arranged, for example, inside the controller 25 of the main body device 3, as shown in FIG. 8.

Each “section” in the present specification is a conceptual matter corresponding to the respective function of the embodiment, and is not always in one-to-one correspondence with specific hardware or a software routine. Accordingly, in the present specification, the embodiment is described assuming a virtual circuit block (section) having the respective function of the embodiment. Also, respective steps of respective procedures in the present embodiment may be executed in different execution order, or simultaneously, or in a different order at each execution, as long as such execution is not against the nature of the respective steps. Furthermore, some or all of the respective steps of the respective procedures in the present embodiment may be realized by hardware.

Although some embodiments of the present invention have been described, the embodiments are illustrated as examples, and do not intend to limit the scope of the invention. The novel embodiments can be carried out in other various modes, and various omissions, replacements and modifications can be made within the scope of the gist of the present invention. The embodiments and modifications are included in the scope and the gist of the invention, and are also included in the invention described in the claims and equivalents of the invention.

According to the scanning endoscope system of the present invention, the amplitude of a fiber may be stably controlled regardless of a change in the environment, by identifying a frequency domain which is not easily affected by a shift of frequency characteristics caused by a change in the environment and by using the frequency domain for a drive frequency.

The present invention is not limited to the embodiment described above, and various modifications and changes may be made within the range of the gist of the present invention.

What is claimed is:

1. A scanning endoscope system comprising:
a scanning section that includes a light guide section that is configured to guide illumination light for illuminating a subject and to emit the illumination light from an emitting end, and an actuator that is configured to swing the emitting end of the light guide section according to a voltage or a current of an electrical signal that is applied to cause the illumination light to scan the subject; and
an application section configured to apply, to the actuator, the electrical signal that takes, as a drive frequency, a frequency at which an amount of change in amplitude at a time of swinging of the emitting end of the light guide section is at or below a predetermined value even when frequency characteristics of the amplitude are changed due to a change in a use condition of the scanning section.

2. The scanning endoscope system according to claim 1, wherein the drive frequency of the electrical signal that is applied to the scanning section by the application section is a frequency at which a ratio of an amount of change in the amplitude to an amount of change in a frequency of the electrical signal that is applied to the actuator is at or below a first threshold that is set, according to the frequency characteristics of the amplitude.

3. The scanning endoscope system according to claim 2, further comprising:
a calculation section configured to acquire frequency characteristics by detecting the amplitude while successively changing, and applying to the actuator, the frequency of the electrical signal, to calculate the ratio of an amount of change in the amplitude to an amount of change in the frequency of the electrical signal that is applied to the actuator, by using the frequency characteristics, and to calculate frequencies at which the ratio is at or below the first threshold as the drive frequency domain; and
a setting section configured to set the drive frequency that is applied to the actuator in the drive frequency domain,
wherein the application section applies the electrical signal having the drive frequency that is set by the setting section to the actuator.

4. The scanning endoscope system according to claim 3, wherein the calculation section does not calculate the ratio for a frequency domain near a resonance frequency.

5. The scanning endoscope system according to claim 3, wherein, with respect to a frequency domain of a predetermined range, if the ratio is continuously at or below the first threshold, the calculation section takes frequencies that are at or below the first threshold as the drive frequency domain.

6. The scanning endoscope system according to claim 2, wherein
the scanning section has a characteristic that the resonance frequency according to the frequency characteristics of the amplitude shifts to a low-temperature side due to a change in the use condition, and
the application section applies, to the actuator, the electrical signal that takes, as the drive frequency, a frequency on a higher frequency side than the resonance frequency, among frequencies at which the ratio is in a range of the first threshold that is set.

7. The scanning endoscope system according to claim 2, wherein the first threshold is substantially zero.

8. The scanning endoscope system according to claim 1, wherein the drive frequency of the electrical signal that is applied to the actuator by the application section is a frequency in a range at or below a second threshold where a proportion of an amount of change in the amplitude is set such that an angle of view of the illumination light is within a set target range even when the frequency characteristics of the amplitude at a time of swinging of the emitting end of the light guide section is changed due to a change in the use condition of the scanning section.

9. The scanning endoscope system according to claim 8, wherein
the scanning section has a characteristic that the resonance frequency according to the frequency characteristics of the amplitude shifts to a low-temperature side due to a change in the use condition, and
the application section applies, to the actuator, the electrical signal that takes, as the drive frequency, a frequency on a higher frequency side than the resonance frequency, among frequencies at which a proportion of the amount of change in the amplitude is at or below the second threshold.
